(19) United States
(12) Patent Application Publication
(10) Pub. No.: US 2026/0207950 A1
(43) Pub. Date: Jul. 23, 2026

(54) IMPLANTABLE MEDICAL DEVICE WITH DISTAL ELECTRODE ARRANGEMENT

(71) Applicant: Medtronic, Inc., Minneapolis, MN (US)

(72) Inventors: Ronson L. Yong, Shoreview, MN (US); Jason D. Hamack, Ramsey, MN (US)

(21) Appl. No.: 19/140,173

(22) PCT Filed: Dec. 14, 2023
(86) PCT No.: PCT/IB2023/062683

§ 371 Date: Jun. 17, 2025

Related U.S. Application Data
(60) Provisional application No. 63/476,852, filed on Dec. 22, 2022.

(51) Int. Cl. A61N 1/375; A61N 1/02; A61N 1/05; A61N 1/368

(52) U.S. Cl. CPC A61N 1/3756; A61N 1/025; A61N 1/0573; A61N 1/368; A61N 1/37512; A61N 1/37518; A61N 1/3758

(57) ABSTRACT

A device comprising: an elongated housing configured to be implanted wholly within a first chamber of a heart, the first chamber of the heart having wall tissue; a distal electrode extending distally from a distal end of the elongated housing, and configured to penetrate into wall tissue of a second chamber of the heart; a reference electrode extending from the distal end of the elongated housing; one or more proximal electrodes extending from the distal end of the elongated housing and separate from the distal electrode and the reference electrode; and processing circuitry within the elongated housing. The processing circuitry is coupled to the distal electrode, the reference electrode, and the one or more proximal electrodes and is configured to sense electrical signals of the heart and pace via the distal electrode, the reference electrode, and the one or more proximal electrodes.

This application claims the benefit of U.S. Provisional Patent Application Ser. No. 63/476,852, filed Dec. 22, 2022, the entire content of which is incorporated herein by reference.

### TECHNICAL FIELD

The disclosure relates to medical devices, and more particularly to configuration of electrodes of medical devices.

### BACKGROUND

Various types of implantable medical devices (IMDs) have been implanted for treating or monitoring one or more conditions of a patient. Such IMDs may be adapted to monitor or treat conditions or functions relating to heart, muscle, nerve, brain, stomach, endocrine organs or other organs and their related functions. Such IMDs may be associated with leads that position electrodes at a desired location, or may be leadless with electrodes integrated with and/or attached to the device housing. These IMDs may have the ability to wirelessly transmit data either to another device implanted in the patient or to another instrument located externally of the patient, or both.

A cardiac pacemaker is an IMD configured to deliver cardiac pacing therapy to restore a more normal heart rhythm. Such IMDs sense the electrical activity of the heart, and deliver cardiac pacing based on the sensed electrical activity, via electrodes. Some cardiac pacemakers are implanted a distance from the heart and coupled to one or more leads that intravascularly extend into the heart to position electrodes with respect to cardiac tissue. Some cardiac pacemakers are sized to be completely implanted within one of the chambers of the heart and may include electrodes integrated with or attached to the device housing rather than leads. Some cardiac pacemakers provide dual chamber functionality, by sensing and/or stimulating the activity of both atria and ventricles, or other multi-chamber functionality. A cardiac pacemaker may provide multi-chamber functionality via leads that extend to respective heart chambers, or multiple cardiac pacemakers may provide multi-chamber functionality by being implanted in respective chambers.

### SUMMARY

In general, this disclosure is directed to implantable medical devices (IMDs) configured to sense electrical signals via a variety of vectors in a three-dimensional (3D) space in tissue of a patient given a single fixed implant orientation. More particularly, this disclosure is directed to IMDs having a plurality of electrodes and a reference electrode configured to define orthogonal bipole vectors similar to a Cartesian coordinate system. Each of the plurality of electrodes may define a reference axis with the reference electrode. The IMD may select a sensing vector from the individual reference axes or from computed combinations of the reference axes and sense electrical signals along the selected sensing vector. Based on the sensed electrical signals, the IMD may deliver electrical stimulation to the tissue of the patient via the plurality of electrodes.

In some examples, a single IMD is implanted in one chamber of a heart of the patient and is able to sense in and/or deliver cardiac pacing to more than one chamber, which may avoid the need for a leaded device or multiple smaller devices to provide such functionality, which may reduce the amount of material implanted within the patient. In some examples, such an implantable medical device includes a first electrode that is configured to penetrate through wall tissue of the heart chamber in which the device is implanted, and into wall tissue of another heart chamber. In addition to the first electrode, the device includes a reference electrode and one or more second electrodes configure to contact the wall tissue of the heart chamber. The electrodes can be connected to a distal end of the device. The first electrode may be a helix configured to penetrate tissue of the patient. Each of the first electrode and the second electrodes may define a reference axis with the reference electrode. Together, the reference axes define a 3D coordinate system within which the IMD may select sensing vectors and pacing vectors. The IMD may sense electrical signals of the heart and deliver cardiac pacing to cardiac tissue in one or more chambers of the heart based on sensing vectors and pacing vectors, respectively. Any sensed signals in the cardiac tissue may be represented in the 3D coordinate system by one or more sensing vectors. The IMD may determine a cardiac pacing therapy for one or more chambers of the heart and deliver the cardiac pacing therapy to the one or more chambers via the first electrode and the second electrodes along a pacing vector within the 3D coordinate system.

In some examples, the example IMDs described herein may improve the accuracy or fidelity of the sensed electrical signal, e.g., by increasing the set of possible sensing vectors available to the IMD. The use of electrodes of the IMD arranged according to define the 3D coordinate system may reduce the reliance on IMD orientation to accurately sense electrical signals, e.g., by allowing the IMD to deviate from a single fixed sensing vector. In some examples, the use of electrodes of the IMD arranged according to define the 3D coordinate system reduces the effect of rotation of the IMD on the sensed electrical signals, e.g., by allowing the IMD to change a sensing vector over time based on signal quality. The use of electrodes of the IMD arranged according to define the 3D coordinate system may also prevent each electrode from sensing cross-contributing and/or overlapping electrical signals along other directions, thereby simplifying and increasing the accuracy of the electrical signal-sensing process. For example, the IMD may sense, along each reference axis of the 3D coordinate system, a unique signal content and determine the electrical signal within the tissue based on the unique signal contents. The IMD may also determine, based on sensing vectors along each reference axis, one or more intermediate sensing vectors and sense electrical signals along the one or more intermediate sensing vectors. The electrode arrangements described herein may also allow the IMD to deliver more accurate stimulation (stimulation that captures intended tissue) and/or minimize unintended stimulation (e.g., cross-chamber stimulation). In some examples, the example IMD may also reduce pacing thresholds required to successfully capture wall tissue of a chamber of the heart.

In one example, this disclosure is directed to a device comprising: an elongated housing that extends from a proximal end of the housing to a distal end of the housing, the elongated housing being configured to be implanted wholly within a first chamber of a heart, the first chamber of the heart having wall tissue; a distal electrode extending distally from the distal end of the elongated housing, the distal electrode being configured to penetrate into wall tissue of a second chamber of the heart that is separate from the first chamber of the heart; a reference electrode extending from the distal end of the elongated housing; one or more proximal electrodes extending from the distal end of the elongated housing, wherein the one or more proximal electrodes are separate from the distal electrode and the reference electrode; sensing circuitry within the elongated housing and coupled to the distal electrode, the reference electrode, and the one or more proximal electrodes; and processing circuitry within the elongated housing, the processing circuitry being configured to control the sensing circuitry to sense electrical signals of the heart via the distal electrode, the reference electrode, and the one or more proximal electrodes.

In some examples, the disclosure describes a method comprising: sensing, by sensing circuitry of an implantable medical device (IMD) and via a plurality of combinations of a distal electrode extending distally from a distal end of an elongated housing of the IMD, a reference electrodes extending from the distal end of the elongated housing, and one or more proximal electrodes extending from the distal end of the elongated housing, a plurality of electrical signals from a heart; determining, by processing circuitry of the IMD and based on the sensed electrical signals and a sensing vector, a combined electrical signal; and delivering, by one or more of the distal electrode and the one or more proximal electrodes, cardiac pacing therapy to one or more chambers of the heart based on the combined signal, wherein the distal electrode is configured to penetrate into the wall tissue of the second chamber, and wherein the one or more proximal electrodes and the reference electrode are configured to maintain contact with the wall tissue of the first chamber without penetration of the wall tissue of the first chamber by the one or more proximal electrodes.

In some examples, the disclosure describes a device comprising: an elongated housing extending from a proximal end to a distal end, the elongated housing being configured to be implanted wholly within a first chamber of a heart; a distal electrode extending distally from the distal end of the elongated housing, the distal electrode being configured to penetrate into wall tissue of a second chamber of the heart, the second chamber of the heart being separated from the first chamber of the heart; two proximal electrodes extending from the distal end of the elongated housing, wherein the two proximal electrodes are separate from the distal electrode, and wherein each proximal electrode of the two proximal electrode is configured to maintain contact with wall tissue of the first chamber without penetration of the wall tissue of the first chamber; a reference electrode extending from the distal end of the elongated housing and separate from the distal electrode and the two or more proximal electrodes, the reference electrode being configured to: define a first axis with the distal electrode; define a second axis with a first proximal electrode of the two proximal electrodes; and define a third axis with a second proximal electrode of the two proximal electrodes; sensing circuitry disposed within the elongated housing; signal generation circuitry disposed within the elongated housing; and processing circuitry within the elongated housing, the processing circuitry being configured to: cause the sensing circuitry to sense electrical signals from the heart along at least two of the first axis, the second axis, and the third axis via the distal electrode, the first proximal electrode, and the second proximal electrode, respectively; determine, based on the sensed electrical signals and a three-dimensional (3D) sensing vector, a combined signal; and cause the signal generation circuitry to deliver cardiac pacing therapy to the heart via one or more of the distal electrode, the first proximal electrode, or the second proximal electrode based on the combined electrical signal.

This summary is intended to provide an overview of the subject matter described in this disclosure. It is not intended to provide an exclusive or exhaustive explanation of the methods and systems described in detail within the accompanying drawings and description below.

### BRIEF DESCRIPTION OF DRAWINGS

The details of one or more examples of this disclosure are set forth in the accompanying drawings and the description below. Other features, objects, and advantages of this disclosure will be apparent from the description and drawings, and from the claims.

FIG. 1 is a conceptual diagram illustrating an example device implanted in the heart of a patient, in accordance with one or more aspects of this disclosure.

FIG. 2A is a perspective diagram illustrating the example device of FIG. 1 with two or more proximal electrodes, in accordance with one or more aspects of this disclosure.

FIG. 2B is a perspective diagram illustrating the example device of FIG. 1 with one proximal electrode, in accordance with one or more aspects of this disclosure.

FIG. 3 is a functional block diagram illustrating an example configuration of the IMD of FIGS. 1-2B, in accordance with one or more aspects of this disclosure.

FIG. 4 is a conceptual diagram of the device of FIGS. 1-3 implanted at a target implant site.

FIG. 5 is a partial view of the device of FIG. 1, in accordance with one or more aspects of this disclosure.

FIG. 6 is a top-down partial view of the device of FIG. 1, in accordance with one or more aspects of this disclosure.

FIG. 7 is a side view of a distal portion of the device of FIG. 1, in accordance with one or more aspects of this disclosure.

FIG. 8 is a flow diagram illustrating an example process for sensing a cardiac electrical signal and delivering cardiac pacing therapy to a heart of a patient via an example device of any of FIGS. 1-7.

### DETAILED DESCRIPTION

In general, this disclosure is directed to configurations of electrodes of implantable medical devices (IMDs). More particularly, this disclosure is directed to IMDs having a plurality of electrodes configured to sense electrical signals from and to deliver electrical stimulation (e.g., cardiac pacing) to tissue of a patient. A physical arrangement of plurality of electrodes on the IMD may define a plurality of reference axes defining a three-dimensional (3D) coordinate system.

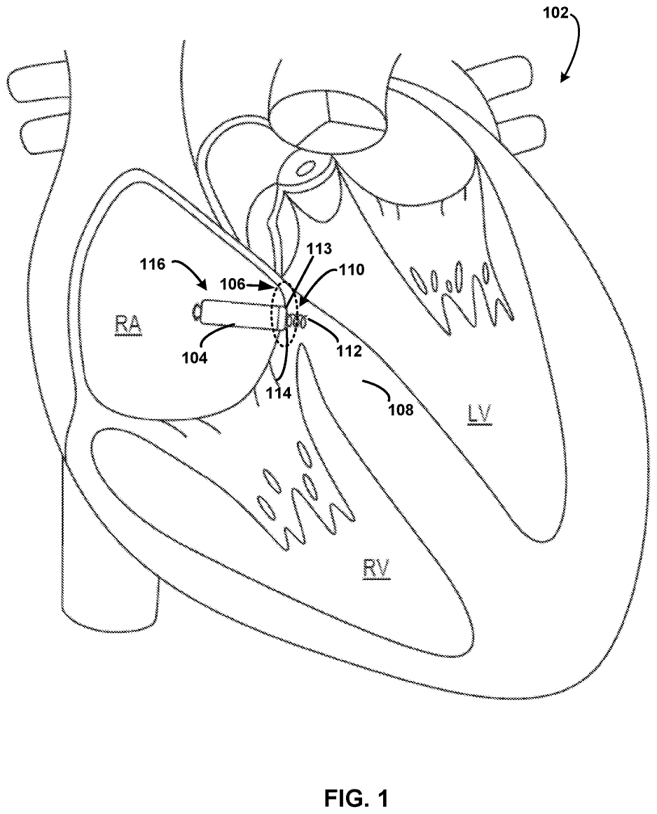

FIG. 1 is a conceptual diagram illustrating an example device 104 implanted in the heart 102 of a patient, in accordance with one or more aspects of this disclosure. Device 104 is shown implanted in the right atrium (RA) of the patient's heart 102 in a target implant region 106, such as the triangle of Koch, in heart 102 with a distal end of device 104 directed toward the left ventricle (LV) of the patient's heart 102. Although in the example of FIG. 1 the distal end of device 104 is directed toward the LV, the distal end may be directed to other targets, such as interventricular septum of heart 102. Target implant region 106 may lie between the bundle of His and the coronary sinus and may be adjacent the tricuspid valve.

Device 104 includes a distal end 110 and a proximal end 116. Distal end 110 includes a distal electrode 112, reference electrode 113, and one or more proximal electrodes 114. Distal electrode 112 may define a helical shape, e.g., as illustrated in FIG. 1. Distal electrode 112 extends from distal end 110 and may penetrate through the wall tissue of a first chamber (e.g., the RA in the illustrated example) into wall tissue of a second chamber (e.g., ventricular myocardium 108 of the LV in the illustrated example). Reference electrode 113 and proximal electrodes 114 may contact the wall tissue of the first chamber as distal electrode 112 penetrates the wall tissue of the first chamber. Reference electrode 113 and proximal electrodes 114 may be disposed at respective positions on distal end 110 of device 104, e.g., around a circumference of distal end 110.

The configuration of electrodes 112, 113, and 114 illustrated in FIG. 1 allows device 104 to sense cardiac signals and/or deliver cardiac pacing to multiple chambers of heart 102, e.g., the RA and ventricle(s) in the illustrated example. In this manner, the configuration of electrodes 112, 113, and 114 may facilitate the delivery of A-V synchronous pacing by single device 104 implanted within the single chamber, e.g., the RA. While device 104 is implanted at target implant region 106 to sense in and/or pace the RA and ventricle(s) in the example shown in FIG. 1, a device having an electrode configuration in accordance with the examples of this disclosure may be implanted at any of a variety of locations to sense in and/or pace any one, two or more chambers of heart 102. For example, device 104 may be implanted at region 106 or another region, and first electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue.

Electrodes 112, 113, and 114 may define a 3D coordinate system. Each of electrodes 112 and 114 may, in combination with reference electrode 113, define a reference axis and sense signal components of an electrical signal in cardiac tissue along the reference axis. For example, a first proximal electrode 114 and reference electrode 113 may define an X-axis of the 3D coordinate system, a second proximal electrode 114 and reference electrode 113 may define a Y-axis of the 3D coordinate system, and distal electrode 112 and reference electrode 113 may define a Z-axis of the 3D coordinate system. Each reference axis may be orthogonal to every other reference axis. For example, the X-axis, Y-axis, and Z-axis defined by electrodes 112, 113, and 114 may be orthogonal relative to each other. Device 104 may define and select sensing vectors based on combinations of the reference axes. Device 104 may then sense electrical signals within wall tissue along the sensing vectors. Any electrical signals (e.g., electrogram (EGM) signals) within wall tissue near target implant region 106 may be sensed by device 104 along one or more sensing vectors and/or along a combination of sensing vectors. Based on the sensed electrical signals, device 104 may determine whether an event has occurred and/or whether heart 102 is experiencing a cardiac condition. Based on the determination, device 104 may then deliver cardiac pacing therapy to one or more chambers of heart 102 along one or more pacing vectors within the 3D coordinate system.

The electrode configuration described herein may provide several advantages over other IMD designs. The use of distal electrode 112 and two or more proximal electrodes 114 to define a 3D coordinate system with reference electrode 113 allows for sensing of signal components by each of distal electrode 112 and proximal electrodes 114 along a single axis without overlap and/or cross-contribution by electrical signals along other axes, thereby simplifying the sensing vector generation and selection process. By allowing selection from a plurality of sensing vectors, including virtual sensing vectors that are based on a combination of two or more physical vectors, the use of the 3D coordinate system may further eliminate a need to precisely orient device 104 during implantation to generate accurate and/or consistent sensing vectors. The increased consistency in the sensed electrical signals may cause device 104 to improve determination of signal amplitude, location, and/or morphology, thereby improving event and/or condition detection capabilities of device 104.

Additionally, placement of reference electrode 113 at distal end 110 provides several advantages. Placement of reference 113 at distal end 110 reduces bipolar electrode spacing between distal electrode 112 and reference electrode 113 and between each proximal electrode 114 and reference electrode 113, e.g., relative to devices for which a reference electrode is located proximal of the distal end. The reduction of bipolar electrode spacing may reduce and/or prevent the sensing of far-field signals (e.g., far-field P waves in ventricular signals, far-field R waves in atrial signals). In some examples, for delivery of pacing or other stimulation, the reduced bipolar electrode spacing may reduce a likelihood of unintended cross-chamber stimulation, e.g., by reduce pacing capture thresholds and voltage thresholds required to stimulate cardiac tissue. In addition, the reduction of pacing capture thresholds and voltage thresholds may reduce power consumption, thereby increase power longevity of device 104.

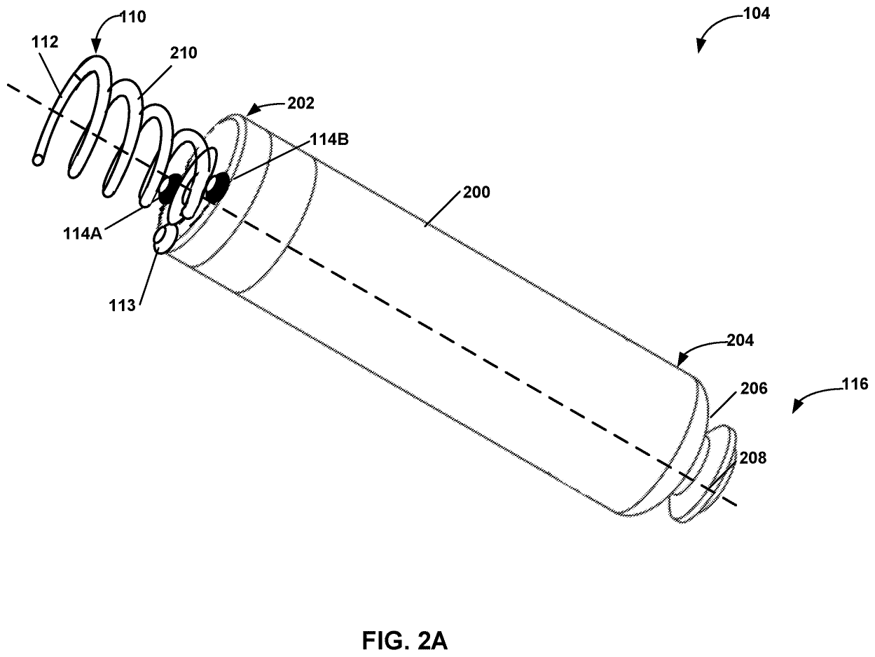

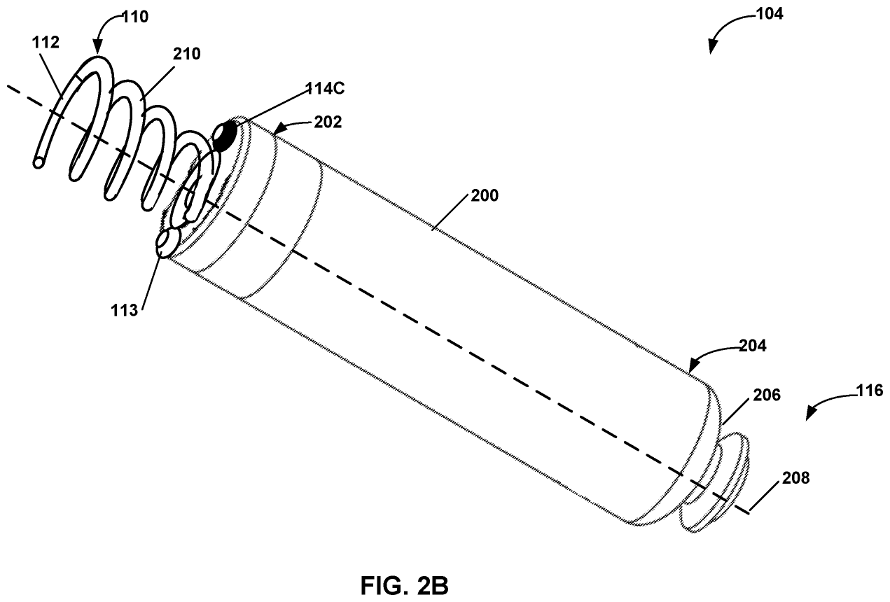

FIG. 2A is a perspective diagram illustrating device 104. Device 104 includes a housing 200 that defines a hermetically sealed internal cavity. Housing 202 may be formed from a conductive material including titanium or titanium alloy, stainless steel, MP35N (a non-magnetic nickel-cobalt-chromium-molybdenum alloy), platinum alloy or other bio-compatible metal or metal alloy, or other suitable conductive material. In some examples, housing 200 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Housing 200 extends between distal end 202 and proximal end 204. In some examples, housing 200 can be cylindrical or substantially cylindrical but may be other shapes, e.g., prismatic, or other geometric shapes. Housing 200 may include a delivery tool interface member 206, e.g., at proximal end 204, for engaging with a delivery tool during implantation of device 104. At distal end 202, housing 200 may define a face of housing 200. The face of housing 200 may be at least substantially orthogonal to longitudinal axis 208. Reference electrode 113 and proximal electrodes 114A, 114B (collectively referred to as “proximal electrodes 114”) are disposed on the face of housing 200 (e.g., around a circumference of housing 200) and extend distally along longitudinal axis 208.

Each of distal electrode 112, reference electrode 113, and proximal electrodes 114 is attached to housing 200 at or near distal end 202. Distal electrode 112 may be disposed on a distal end of elongated body 210 extending distally from distal end 202 of housing 200. Elongated body 210 may extend from a first end fixedly attached to housing 200 at or near distal end 202 to a second end that, in the example of FIG. 2A, is not attached to housing 202 other than via the first end (e.g., is a free end). The second end of elongated body 210 may retain or define distal electrode 112. Elongated body 210 may extend along longitudinal axis 208 and may define a helical and/or spiral shape or any other shape.

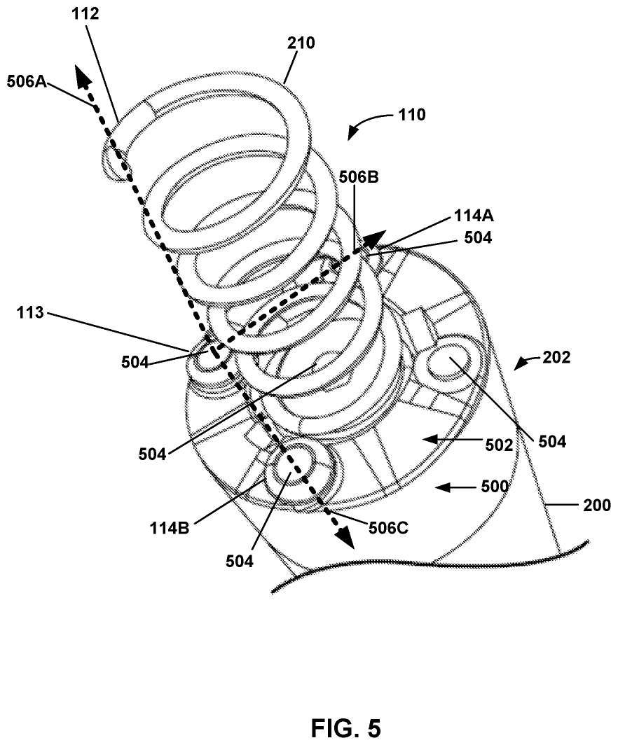

Elongated body 210 may include one or more coatings (e.g., electrically insulative coating(s)) configured to define a distal electrically active region or distal electrode 112. In some examples, the distal electrically active region may be more proximate to the second, e.g., distal, end of elongated body 210. In the example of FIG. 2A, the distal electrically active region includes the distal end of elongated body 210. Each of reference electrode 113 and/or proximal electrodes 114 may include one or more coatings configured to define a corresponding electrically active region on an outer surface of the respective electrode. In some examples, as illustrated in FIG. 2A, the electrical active regions of reference electrode 113 and/or proximal electrodes 114 forms a ring around a steroid eluting element or a therapeutic substance dispensing devices, e.g., as discussed in greater detail in with respect to FIG. 5. Each electrode of reference electrode 113 and/or proximal electrodes 114 may be a button electrode, a spring electrode, or any other suitable type or shape of electrode.

Each electrode of electrodes 112, 113, and 114 may be formed of an electrically conductive material, such as titanium, platinum, iridium, tantalum, stainless steel or alloys thereof. Each electrode of electrodes 112, 113, and 114 may be coated with an electrically insulating coating, e.g., a parylene, polyurethane, silicone, epoxy, or other insulating coating, to reduce the electrically conductive active surface area of the respective electrode and define a corresponding electrically active region. Defining electrically active regions of electrodes 112, 113, and 114 by covering portions of each electrode with an insulating coating may increase the electrical impedance of electrodes 112, 113, and 114 and thereby reduce the current delivered during a pacing pulse that captures the cardiac tissue. A lower current drain conserves the power source, e.g., one or more rechargeable or non-rechargeable batteries, of device 104.

In some examples, each of electrodes 112, 113, and 114 may have an electrically conducting material coating on the corresponding electrically active regions. For example, the electrically active regions of any of electrodes 112, 113, and 114 may be coated with titanium nitride (TiN). Each of electrodes 112, 113, and 114 may be made of substantially similar material or may be made of different material from one another.

In the example of FIG. 2A, elongated body 210 takes the form of a helix. In some examples, distal electrode 112 may be an elongated body defining a helix. In some examples, a helix is an object having a three-dimensional shape like that of a wire wound uniformly in a single layer around a cylindrical or conical surface or mandrel such that the wire would be in a straight line if the surface were unrolled into a plane. Reference electrode 113 and proximal electrodes 114 are disposed on distal end 202 and each may include a button electrode, e.g., as illustrated in FIG. 2A, or any other suitable type or shape of electrode.

In some examples, as illustrated in FIG. 2A, device 104 may have two proximal electrodes 114 disposed on distal end 202 of housing 200. In other examples, e.g., as illustrated and described in greater detail in FIG. 2B, device 104 may have one proximal electrode 114 disposed on distal end 202. Each of proximal electrodes 114 may define a reference axis with reference electrode 113. For example, proximal electrode 114A may define a first reference axis with reference electrode 113 and proximal electrode 114B may define a second reference axis with reference electrode 113. Proximal electrodes 114 and reference electrode 113 may be arranged around a circumference of distal end 202 such that the first reference axis is orthogonal to the second reference axis. In some examples, device 104 may include three or more proximal electrodes 114 disposed on distal end 202 of housing 200. In such an example, any selection of proximal electrodes 112 of the three or more proximal electrodes 114 may define separate reference axes with reference electrode 113. Reference electrode 113 and proximal electrodes 114 may be equally spaced around the circumference of distal end 202. In some examples, one or more of reference electrode 113 and proximal electrodes 114 may be disposed at a user-selected angle away from a proximal end of elongated body 210.

Elongated body 210 may be formed of an electrically conductive material, such as titanium, platinum, iridium, tantalum, or alloys thereof, and/or of electrically nonconductive material(s). At least portions of elongated body 210 (e.g., a portion proximal to distal electrode 112) may be coated with an electrically insulating coating, e.g., a parylene, polyurethane, silicone, epoxy, or other insulating coating. In some examples, elongated body 210 may be formed from a memory metal (e.g., Nitinol, platinum, titanium, MP35N, or the like) and/or a memory polymer (e.g., silicone, polyurethane, polyether ether ketone (PEEK), or the like), or other materials.

In some examples, elongated body 210 may include one or more anti-rotation features. The anti-rotation features may include a shape of elongated body 210, dimensions (e.g., outer diameter, pitch, or the like) of elongated body 210, one or more features disposed on an outer surface of elongated body 210, or the like. The shape and/or dimensions of elongated body 210 may include a geometric shape of elongated body 210, a varying diameter configuration of elongated body 210, a varying pitch configuration of elongated body 210, a waveform configuration of elongated body 210, or any combination herein. The one or more anti-rotation features disposed on elongated body 210 may include, but are not limited to, elongate darts, barbs, or tines. The one or more anti-rotation features may resist rotation of elongated body 210 and/or distal electrode 112 (e.g., by penetrating the tissue, by increasing the friction between elongated body 210 and the tissue, or the like). The one or more anti-rotation features may be disposed on any quadrant of the face of distal end 202 except a quadrant containing a proximal end of elongated body 210, e.g., to stabilize device 104 within the tissue.

In some examples, electrodes 113 and 114 are positioned on distal end 202 of housing 200 and resist rotation of elongated body 210 and/or distal electrode 112. Each of electrodes 113 and 114 may be at least partially enveloped by the tissue of heart 102 and engage with the tissue to prevent rotation of elongated body 210 and/or distal electrode 112. In some examples, one or more of electrodes 113 and 114 is disposed in a quadrant of the face of distal end 202 not containing a proximal end of elongated body 210 or is separated from the proximal end of elongated body 210 by a predetermined angle, e.g., to prevent unintended rotation of elongated body 210.

In some examples elongated body 210 may define a helix and/or spiral having a varying diameter configuration, e.g., to place distal electrode 112 at a same radial location as reference electrode 113 relative to longitudinal axis 208 and at a more distal longitudinal position than reference electrode 113 relative to longitudinal axis 208. In such examples, distal electrode 112 may define a reference axis with reference electrode 113 that is orthogonal to distal end 202 of housing 200 and parallel to longitudinal axis 208. In some examples, elongated body 210 may define a helix and/or spiral having an outer diameter at a proximal end of elongated body 210 that is smaller than the outer diameter at a distal end of elongated body 210, e.g., at distal electrode 112. The varying diameter may cause elongated body 210 to resist rotation within the tissue of heart 102.

As illustrated in FIG. 2A, elongated body 210 may be a right-hand wound helix, although in other examples distal electrode 112 may have a left-hand wound helix. Elongated body 210 may have a constant or varying pitch along longitudinal axis 208. In some example, elongated body 210 may have a shape other than helical. For example, elongated body 210 may have a geometrical shape (e.g., a triangular shape, a rectangular shape, a hexagonal shape, an octagonal shape, a lobed shape, or the like). Such a geometrical shape may be equilateral.

Each of distal electrode 112, reference electrode 113, and/or proximal electrodes 114 may vary in size and shape in order to enhance tissue contact of electrically active region(s) defined by any of electrodes 112, 113, and 114 with tissue of heart 102. For example, distal electrodes 112 can have a round cross-section or could be made with a flatter cross-section (e.g., oval or rectangular) based on tissue contact specifications. In some examples, one or more of reference electrode 113 and proximal electrodes 114 may have an outer surface that varies in size and shape (e.g., an oval outer surface, an outer surface with a larger diameter, or the like) in order to enhance tissue contact of the corresponding electrically active regions. In some examples, as illustrated in FIG. 2A, reference electrode 113 and proximal electrodes 114 may be disposed directly on distal end 202 of housing 200.

A distal end of distal electrode 112 may have a conical, hemi-spherical, or slanted edge distal tip with a narrow tip diameter, e.g., less than 1 millimeter (mm), for penetrating into and through tissue layers. In some examples, the distal end of distal electrode 112 can be a sharpened or angular tip or sharpened or beveled edges, but the degree of sharpness may be constrained to avoid a cutting action that could lead to lateral displacement of the distal end of distal electrode 112 and undesired tissue trauma. In some examples, elongated body 210 may have a maximum diameter at its base that interfaces with housing distal end 202. In such examples, the outer diameter of the helix defined by elongated body 210 may decrease from housing distal end 202 to the distal end of distal electrode 112. In some examples, the diameter of distal electrode 112 may vary from housing distal end 204 to the distal end of distal electrode 112.

In some examples, two or more of electrodes 112, 113, and 114 may be used to sense electrical signals from heart 102 at target implant region 106. Distal electrode 112 and proximal electrodes 114 may define three reference axes with reference electrode 113 and the three reference axes may define a 3D coordinate system encompassing target implant region 106. When device 106 senses an electrical signal (e.g., an atrial signal, a ventricular signal) within target implant region 106, each of distal electrode 112 and proximal electrodes 114 may sense a component of the electrical signal along the corresponding reference axis. Device 104 may then represent the sensed electrical signal as a vector comprising one or more sensing vectors. Device 104 may determine, based on the sensed components and a sensing vector, an occurrence of an event (e.g., depolarization) or a cardiac condition in one or more chamber of heart 102. Similarly, device 104 may determine a pacing vector within 3D space defined by the 3D coordinate system, the pacing vector being configured to deliver cardiac pacing to one or more chambers of heart 102. Related to the foregoing, the inventors have found that it is desirable to configure the electrodes so as to maximize the amount or volume of viable tissue between the electrodes. Embodiments disclosed herein accomplish this within the context of an electrode array located on the distal end of an elongate device housing.

A sensing vector is a vector used by device 104 to sense atrial signals or ventricular signals from target implant region 106. For example, device 104 may sense electrical signals from target implant region 106 along a plurality of sensing vectors to sense electrical signals at corresponding locations within target implant region 106 (e.g., atrial myocardium, ventricular myocardium) Device 104 may define sensing vectors within the 3D space defined by the 3D coordinate system. Similarly, a pacing vector is a vector used by device 104 to deliver cardiac pacing to atrial myocardium or ventricular myocardium and may be defined within the 3D space. Use of electrodes 112, 113, and 114 to define pacing vectors and sensing vectors may reduce the significance of the (sometimes unredictable) orientation of device 104 during implantation and improve pacing and/or sensing quality, e.g., due to a reduction and/or elimination of overlapping signal components from another region within target implant region 106. For example, each of electrodes 112 and 114 would only sense components of electrical signals along a single axis (e.g., the reference axis) which eliminates the need to separate the cross-contribution of components of the electrical signals along multiple axes to determine position of the sensing vector relative to device 104.

In some examples, proximal electrodes 114 may be configured as atrial cathode electrodes for delivering pacing pulses to the atrial tissue, e.g., at target implant region 106 in combination with reference electrode 113. Proximal electrodes 114 and reference electrode 113 may also be used to sense atrial P-waves for use in controlling atrial pacing pulses (delivered in the absence of a sensed P-wave) and for controlling atrial-synchronized ventricular pacing pulses delivered using distal electrode 112 as a cathode and reference electrode 113 as the return anode.

Placement of reference electrode 113 at distal end 202 reduces a bipolar electrode spacing between proximal electrodes 114 and reference electrode 113 and between distal electrode 112 and reference electrode 113, e.g., relative to other IMDs with a reference electrode disposed proximally on housing 202. The reduced bipolar electrode spacing may improve sensing and pacing capabilities of electrodes 112, 113, and 114. For example, electrodes 112 and 114 may deliver cardiac pacing therapy to atrial myocardium and/or ventricular myocardium at lower voltages, which may reduce a likelihood for unintended cross-chamber capture of cardiac tissue. Electrodes 112 and 114 may deliver cardiac pacing to atrial myocardium and ventricular myocardium with reduced pacing thresholds, e.g., due to an increase in surface area of electrodes (e.g., of proximal electrodes 114), due to a reduction in impedance, and/or due to the reduced bipolar electrode spacing. For example, the reduction in bipolar electrode spacing increases current density of a pacing pulse delivered by device 104 to the atrial myocardium and/or the ventricular myocardium, thereby reducing pacing thresholds for the atrial myocardium and/or the ventricular myocardium, respectively.

At distal end 110, device 104 includes a distal fixation assembly including distal electrode 112, reference electrode 113, proximal electrodes 114, elongated body 210, and housing distal end 202. A distal end of distal electrode 112 can be configured to rest within a ventricular myocardium of the patient, and reference electrode 113 and proximal electrodes 114 can be configured to contact an atrial endocardium of the patient. Proximal electrodes 114 may be individually selectively coupled to sensing and/or pacing circuitry enclosed by housing 200 for use as an anode with distal electrode 112 or as an atrial cathode electrode, or may be electrically common and not individually selectable. In some examples, each of distal electrode 112 and proximal electrodes 114 may be coupled to sensing and/or pacing circuitry within housing 200 by a separate feedthrough or feedthrough assembly.

During implantation of device 104 within target implant region 106, a clinician may sense signals from tissue of heart 102 at target implant region 106 via each of electrodes 112 and 114, e.g., to determine a location and orientation of device 104 within target implantation region 106. The clinician may orient device 104 within target implant region 106 based on the sensed signals from electrodes 112 and 114. For example, the clinician may orient device 104 based on the sensed signals such that a reference axis defined by distal electrode 112 and reference electrode 113 extends distally towards tissue of a second chamber of heart 102 and reference electrode 113 and proximal electrodes 114 define a plane encompassing tissue of the first chamber of heart 102.

Device 104 may auto-rotate, e.g., due to torque on device 104 by tissue of heart 102 and elongated body 210 may partially advance out of tissue of heart 102, leading to a loss of contact between the tissue and at least one of electrodes 113 and 114. In such examples, device 104 may sense signals from (e.g., via far field sensing) and deliver cardiac pacing to the first chamber of heart 102 via distal electrode 112 and reference electrode 113. Device 104 may determine a sensing vector based on a reference axis defined by distal electrode 112 and reference electrode 113 to sense electrical signals from tissue of the first chamber of heart 102.

FIG. 2B is a perspective diagram illustrating the example device of FIG. 1 with one proximal electrode 114C, in accordance with one or more aspects of this disclosure. Proximal electrode 114C may be substantially similar to proximal electrodes 114 (i.e., proximal electrode 114A, proximal electrode 114B). As illustrated in FIG. 2, proximal electrode 114C may define a proximal reference axis with reference electrode 113 and distal electrode 112 may define a distal reference axis with reference electrode 113.

In some examples, as illustrated in FIG. 2B, the proximal reference axis is orthogonal to the distal reference axis. Device 104 may be configured to sense electrical signals in heart 104 along a reference plane defined by the proximal reference axis and the distal reference axis. During implantation, a clinician may rotate device with target implant region 106 to rotate the reference plane about longitudinal axis 208 and place the reference plane at a predetermined orientation within target implant region 106.

During implantation, the clinician may navigate device 104 within the first chamber of heart 102 to map the first chamber and determine a position and intended orientation of device 104 within target implant region 106 of the fist chamber based on the mapping of the first chamber. For example, the clinician may sense, via electrodes 112, 113, and 114 on device 104, depolarization waveform patterns in the tissue of heart 102 and determine a placement and/or orientation of device 104 based on the depolarization waveform patterns. Electrodes 112, 113, and 114 forming reference axes extending in different directions (e.g., orthogonally to other reference axes) may provide the clinician with sensing and mapping capabilities in the different directions at any given time, thereby simplifying the mapping process.

Reference electrode 113 and proximal electrode 114C may be disposed on distal end 202 of housing 200. Placement of distal electrode 112, reference electrode 113, and proximal electrode 114C may cause improved sensing and pacing capabilities and/or a reduction in pacing thresholds, e.g., as described in greater detail above with respect to FIG. 2A.

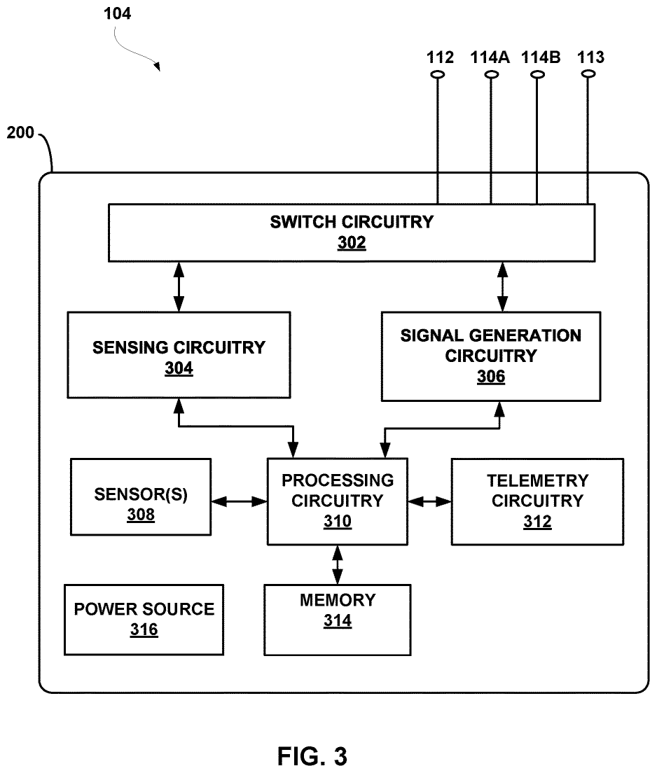

FIG. 3 is a functional block diagram illustrating an example configuration of device 104. As illustrated in FIG. 3, device 104 include electrodes 112, 113, and 114, which may be configured as described with respect to FIGS. 1 and 2. For example, as described with respect to FIGS. 1 and 2, distal electrode 112 may be configured to extend from distal end 202 of housing 200 and may penetrate through the wall tissue of a first chamber (e.g., the RA) into wall tissue of a second chamber (e.g., the LV). Reference electrode 113 and proximal electrodes 114 extend from distal end 202 of housing 200 and may be configured to maintain contact with the wall tissue of the first chamber without penetration of the wall tissue of the first chamber.

In the example shown in FIG. 3, device 104 includes switch circuitry 302, sensing circuitry 304, signal generation circuitry 306, sensor(s) 308, processing circuitry 310, telemetry circuitry 312, memory 314, and power source 316. The various circuitry may be, or include, programmable or fixed function circuitry configured to perform the functions attributed to respective circuitry. Memory 314 may store computer-readable instructions that, when executed by processing circuitry 310, cause device 104 to perform various functions. Memory 314 may be a storage device or other non-transitory medium. The components of device 104 illustrated in FIG. 3 may be housed within housing 202.

Signal generation circuitry 306 generates electrical stimulation signals, e.g., cardiac pacing pulses. Switch circuitry 302 is coupled to electrodes 112, 113, and 114, may include one or more switch arrays, one or more multiplexers, one or more switches (e.g., a switch matrix or other collection of switches), one or more transistors, or other electrical circuitry. Switch circuitry 302 is configured to direct stimulation signals from signal generation circuitry 306 to a selected combination of electrodes 112, 113, and 114, having selected polarities, e.g., to selectively deliver pacing pulses to the RA, the LV, or interventricular septum of heart 102. For example, in order to pace one or both of the ventricles, switch circuitry 302 may couple distal electrode 112, which has penetrated to wall tissue of a ventricle or the intraventricular septum, to signal generation circuitry 306 as a cathode, and one or more of reference electrode 113 or proximal electrodes 114 to signal generation circuitry 306 as an anode. As another example, in order to pace the RA, switch circuitry 302 may couple one or more of proximal electrodes 114 to signal generation circuitry 306 as a cathode, and one or both of distal electrode 112 or reference electrode 113 to signal generation circuitry 306 as an anode. As another example, switch circuitry 302 may alternately couple electrodes 112, 113, and/or 114 to signal generation circuitry 306 as cathodes or anodes to deliver pacing pulses to heart 102.

Switch circuitry 302 may also selectively couple sensing circuitry 304 to selected combinations of electrodes 112, 113, and 114, e.g., to selectively sense the electrical activity of either the RA or ventricles of heart 102. Sensing circuitry 304 may include filters, amplifiers, analog-to-digital converters, or other circuitry configured to sense cardiac electrical signals via electrodes 112, 113, and/or 114. For example, switch circuitry 302 may couple one or more sensing vectors to respective sensing channels provided by sensing circuitry 304 to sense ventricular or atrial cardiac electrical signals. Switch circuitry 302 may then direct signals from one or more of electrodes 112 and 114 composing a sensing vector to the corresponding sensing channel.

In some examples, sensing circuitry 304 may select sensing vectors for the sensing of the electrical activity. Each sensing vector may be a combination of signal components from one or more of electrodes 112 and 114. Sensing circuitry 304 may represent any vector within the 3D coordinate system based on one or more sensing vectors. For example, sensing circuitry 304 determines an optimal sensing vector based on a sensing vector within the 3D coordinate system or a combination of two or more sensing vectors within the 3D coordinate system. Once sensing circuitry 304 selects a sensing vector, sensing circuitry 304 may sense electrical signals from heart 102 along the selected sensing vector to sense cardiac activity of one or more chambers of heart 102. The use of sensing vectors within the 3D coordinate system by sensing circuitry 304 to sense electrograms (EGMs) in cardiac tissue reduces reliance by sensing circuitry 304 on a precise orientation of device 104 (e.g., of electrodes 112, 113, and 114) within target implant region 106 and/or removes the effect of rotation of device 104 on the EGMS, thereby increasing the flexibility of the sensing capabilities of sensing circuitry 304. In some examples, the use of the sensing vectors causes sensing circuitry 304 to sense EGMs with more consistent amplitude and morphology. In some examples, based on changes in orientation of device 104 and/or physiology of heart 102, sensing circuitry 304 may adjust sensing vectors and/or select sensing vectors accordingly, thereby maintaining the quality of the sensed signals.

In some examples, sensing circuitry 304 is configured to detect events, (e.g., depolarizations) and/or cardiac conditions (e.g., presence of arrhythmias, tachycardia, or the like) within the cardiac electrical signals, and provide indications thereof to processing circuitry 310. In this manner, processing circuitry 310 may determine the timing of atrial and ventricular depolarizations, and control the delivery of cardiac pacing, e.g., AV synchronized cardiac pacing, based thereon. Processing circuitry 310 may select pacing vectors within the 3D coordinate system defined by electrodes 112, 113, and 114. Processing circuitry 310 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 310 herein may be embodied as firmware, hardware, software or any combination thereof.

In some examples, sensing circuitry 304 may sense signals along each sensing vector based on sensed signals from electrodes 112 and 114 along corresponding reference axes. Sensing circuitry 304 may then combine the sensed signals to determine the sensed signals along the sensing vector. For each sensing vector, each sensed signal could have a different gain factor used to determine the sensed signals. For example, signals from reference axes defined by proximal electrodes 114 and reference electrode 113 may have a different gain factor than signals from a reference axis defined by distal electrode 112 and reference electrode 113. In some examples, each sensing vector is associated with and sensed by a dedicated sense channel within sensing circuitry 304. In some examples, each sensing vector may be switched, e.g., by switch circuitry 302, to a sense channel within sensing circuitry 302 that have limited functionality.

Sensor(s) 308 may include one or more sensing elements that transduce patient physiological activity to an electrical signal to sense values of a respective patient parameter. Sensor(s) 308 may include one or more accelerometers, optical sensors, chemical sensors, temperature sensors, pressure sensors, or any other types of sensors. Sensor(s) 308 may output patient parameter values that may be used as feedback to control sensing and delivery of therapy by device 104.

Telemetry circuitry 312 supports wireless communication between device 104 and an external programmer (not shown in FIG. 3) or another computing device under the control of processing circuitry 310. Processing circuitry 310 of device 104 may receive, as updates to operational parameters from the computing device, and provide collected data, e.g., sensed heart activity or other patient parameters, via telemetry circuitry 312. Telemetry circuitry 312 may accomplish communication by radiofrequency (RF) communication techniques, e.g., via an antenna (not shown).

Power source 316 delivers operating power to various components of device 104. Power source 316 may include a rechargeable or non-rechargeable battery and a power generation circuit to produce the operating power. Recharging may be accomplished through proximal inductive interaction between an external charger and an inductive charging coil within device 104.

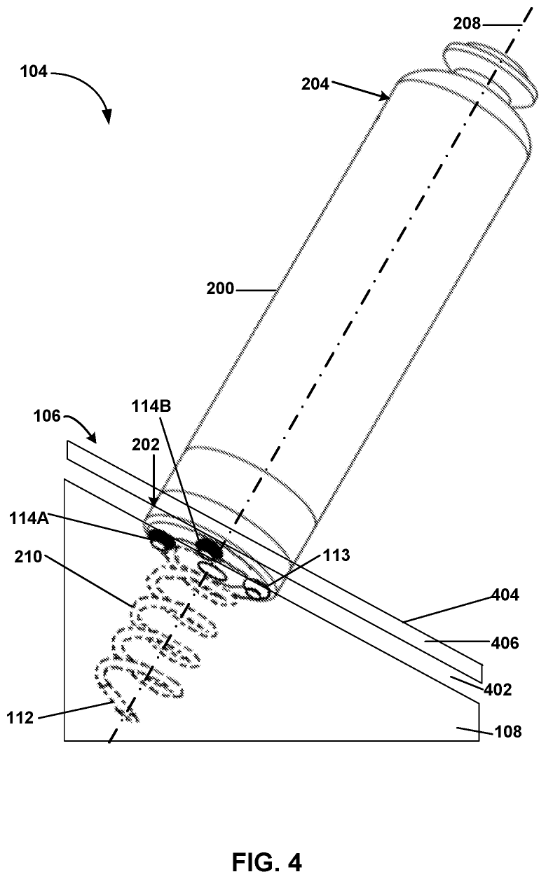

FIG. 4 is a conceptual diagram of device 104 implanted at target implant region 106. distal electrode 112 may be inserted (e.g., in a manner similar to rotating and advancing a threaded screw) such that tissue becomes engaged with elongated body 210. As elongated body 210 becomes engaged with tissue, distal electrode 112 pierces into the tissue at target implant region 106 and advances through atrial myocardium 406 and central fibrous body 402 to position distal electrode 112 in ventricular myocardium 108 as shown in FIG. 4. In some examples, distal electrode 112 penetrates into the interventricular septum. In some examples, distal electrode 112 does not perforate entirely through the ventricular endocardial or epicardial surface.

In some examples, manual pressure applied to the housing proximal end 204, e.g., via an advancement tool, provides the longitudinal force to pierce the cardiac tissue at target implant region 106. In some examples, actuation of an advancement tool rotates elongated body 210 configured as a helix about longitudinal axis 208. The rotation of elongated body 210 about the longitudinal axis 208 advances distal electrode 112 through atrial myocardium 406 and central fibrous body 402 to position distal electrode 112 in ventricular myocardium 108 as shown in FIG. 4.

As distal electrode 112 advances into the tissue, the distance between distal end 202 and atrial endocardium 404 decreases until reference electrode 113 and proximal electrodes 114 contacts, and may press against, the surface of atrial endocardium 404. Electrodes 113 and 114 may press against the surface of atrial endocardium 404 and compress the wall tissue. The compression of the wall tissue may increase friction between electrodes 113 and 114 and the wall tissue and prevent rotation of distal electrode 112 due to movement of tissue of heart 102 (e.g., movement of ventricular myocardium 108, atrial myocardium 406, central fibrous body 402, or the like). Electrodes 113 and 114 pressing against heart tissue may cause heart tissue to become engaged with electrodes 113 and 114. Retraction of electrodes 113 and 114 from the surface of atrial endocardium 404 may be prevented by distal electrode 112.

Target implant region 106 in some pacing applications is along atrial endocardium 404, substantially inferior to the AV node and bundle of His. In some examples, target implant region 106 is within the triangle of Koch. distal electrode 112 can have a length that penetrates through atrial endocardium 404 in target implant region 106, through the central fibrous body 402 and into ventricular myocardium 108 without perforating through the ventricular endocardial surface. In some examples, when the full length of distal electrode 112 is fully advanced into target implant region 106, distal electrically active region 216 rests within ventricular myocardium 108 and reference electrode 113 and proximal electrodes 114 are positioned in intimate contact with atrial endocardium 404. Distal electrode 112 may extend from housing distal end 204 approximately 3 mm to 12 mm in various examples. In some examples, distal electrode 112 may extend a distance from housing 202 of at least 3 millimeters (mm), at least 3 mm but less than 20 mm, less than 15 mm, less than 10 mm, or less than 8 mm in various examples. The diameter of distal electrode 112 may be less than 2 mm and may be 1 mm or less, or even 0.6 mm or less.

FIGS. 5, 6, and 7 are partial views of the device of FIGS. 1-4 from different perspectives, in accordance with one or more aspects of this disclosure. FIG. 5 is a partial view of device 104 of FIG. 1 including distal end 110. Housing 200 includes a header 500. In some examples, header 500 may be separate or integral with housing 200 and can be made of the same or different materials as housing 200. In some examples, as illustrated in FIG. 5, distal end 202 of housing 200 includes a peripheral region, e.g., around distal electrode 112. Reference electrode 113 and proximal electrodes 114 may be disposed in the peripheral region of distal end 202. Each of proximal electrodes 114 are spaced relative to reference electrode 113 to define a reference axis. For example, proximal electrode 114A defines a reference axis 506B (also referred to as “X-axis 506B”) with reference electrode 113 and proximal electrode 114B defines a reference axis 506C (also referred to as “Y-axis 506C) with reference electrode 113. Similarly, distal electrode 112 may define a reference axis 506A (also referred to as “Z-axis 506C”) with reference electrode 113.

In some examples, elongated body 210 may define a helix and/or coil defining a varying outer diameter at a distal end of elongated body 210 than at a proximal end of elongated body 210, e.g., to place distal electrode 112 at a same radial position as reference electrode 113 relative to longitudinal axis 208. By placing distal electrode 112 at the same radial position as reference electrode 113, distal electrode 112 would not sense overlapping signal components along reference axis 506A. Reference electrode 113 and proximal electrodes 114 may define a height extend distally away from distal end 202 of housing 200, e.g., to contact tissue of heart 102. Each of electrodes 113 and 114 may all have a same height, may have a same height as another of electrodes 113 and 114, or may all have different heights, e.g., to define orthogonal reference axes 506. For example, if distal electrode 112 is radially offset from reference electrode 113 (e.g., distal electrode 112 is located in a same circumferential position as reference electrode 113), the heights of reference electrode 113 and of proximal electrodes 114 may be different, e.g., to cause each of the reference axes 506A-506C (collectively referred to as “reference axes 506”) to be orthogonal to every other of reference axes 506.

In some examples, as illustrated in FIG. 5, reference axes 506 define a 3D coordinate system. Each of reference axes 506 may be orthogonal to every other reference axis of reference axes 506. For example, reference axis 506A is orthogonal to reference axis 506B and reference axis 506C, and vice versa. Device 104 may sense electrical signal components of an electrical signal in heart 102 along each of reference axes 506. Device 104 may determine, based on the sensed electrical components, a sensed electrical signal along a sensing vector. Based on the sensed electrical signal along sensing vector(s), device 104 may determine electrical signals within heart 102 and/or whether to deliver cardiac pacing therapy to one or more chambers of heart 102.

Inflammation of patient tissue may result from interaction with device 104. For example, penetration of tissue by distal electrode 112 and/or contact between tissue and reference electrode 113 and/or proximal electrodes 114 may result in inflammation of the tissue. Inflammation of patient tissue proximate to electrodes may result in higher thresholds for stimulation delivered to the tissue to activate, or capture, the tissue. Higher capture thresholds may, in turn, increase the consumption of a power source of device 104 associated with delivery of the stimulation.

In some examples device 104 includes one or more steroid eluting elements 504 (collectively referred to as “steroid eluting elements 504”), e.g., disposed on distal end 202. The steroid may mitigate inflammation of patient tissue resulting from interaction with the IMD. Steroid eluting elements 504 may be configured to elute one or more steroids to tissue in proximity to elements 504 over time. In some examples, steroid eluting elements 504 comprise one or more monolithic controlled release devices (MCRDs). In some examples, steroid eluting elements 504 may be therapeutic substance dispensing devices.

In some examples, device 104 includes one or more steroid eluting elements 504 configured to elute one or more steroids to tissue proximate to distal electrode 112. Steroid eluting elements 504 may be disposed within recess(es) defined by reference electrode 113 and/or proximal electrodes 114. In some examples, steroid eluting element 504 may be disposed at a center of face 502, e.g., within an annulus defined by elongated body 210.

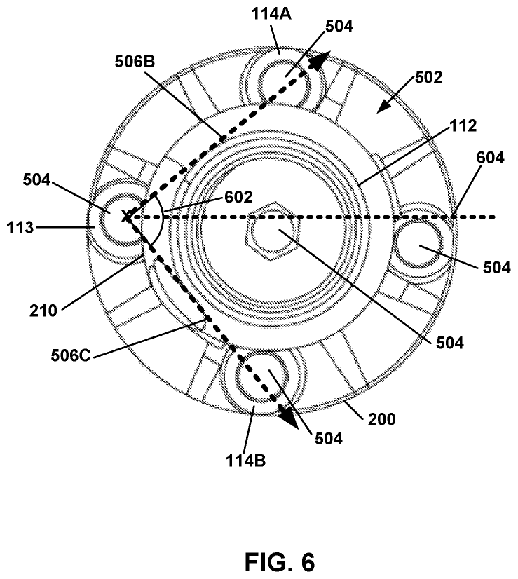

FIG. 6 is a top-down partial view of device 104 and of face 502. As illustrated in FIG. 6, X-axis 506B and Y-axis 506C are separated by angle 602. Device 104 may sense electrical signals (e.g., atrial signals) along sensing vector 604. Sensing vector 604 may be defined by sensed components along X-axis 506B and Y-axis 506C, by proximal electrodes 114A and 114B, respectively. Use of sensing vector 604 by sensing circuitry 304 may prevent the sensing of overlapping signals (e.g., ventricular signals), thereby simplifying detection of EGMs, e.g., from atrium of heart 102. For example, sensing, by sensing circuitry 304 and via proximal electrodes 114, along sensing vector 604 prevents sensing of electrical signals in the direction of Z-axis 506A. In some examples, X-axis 506B and Y-axis 506C may be orthogonal to each other, e.g., to prevent the sensing of overlapping signals by each of electrodes 114 of electrical signals along the other axes of reference axis 506. For example, when X-axis 506B and Y-axis 506C are orthogonal, proximal electrode 114A may not sense electrical signals in the direction of Y-axis 506C. In such examples, angle 602 is 90 degrees. In some examples, X-axis 506B and Y-axis 506C are substantially orthogonal (e.g., angle 602 is between about 80 degrees to 100 degrees), e.g., to minimize the sensing of overlapping signals along Z-axis 506A by electrodes 114.

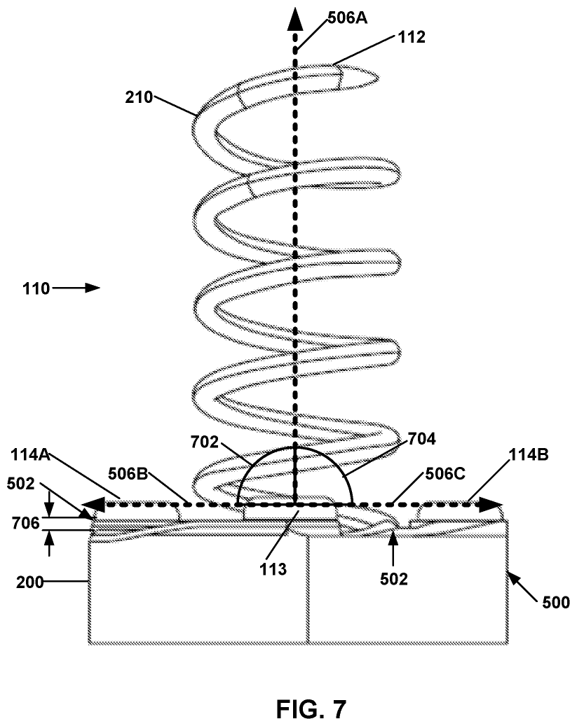

FIG. 7 is a side view of distal end 204 of device 104. As illustrated in FIG. 7, Z-axis 506A may be separated from X-axis 506B by angle 702 and from Y-axis 506C by angle 704. In some examples, Z-axis 506A may be orthogonal to both X-axis 506B and Y-axis 506C, e.g., to prevent sensing by distal electrode 112 of electrical signals along X-axis 506B and/or Y-axis 506C. In such examples angles 702 and 704 are 90 degrees. In some examples, X-axis 506B, Y-axis 506C, and Z-axis 506A are substantially orthogonal to each other. In such examples, angles 702 and 704 is between about 80 degrees to 100 degrees.

In some examples, as illustrated in FIG. 7, each of reference electrode 113 and proximal electrodes 114 are offset from face 502 by a distance 706, e.g., to improve contact with wall tissue of the first chamber and/or sensing and/or pacing capabilities of electrodes 113 and 114. Distance 706 may be between 0.34556 millimeters (mm) to about 1.27 mm (e.g., about 0.014 inches (in) to about 0.05 in). Each of electrodes 113 and 114 may be offset from face 502 by a same distance 706, two or more of electrodes 113 and 114 may be offset from face 502 by a same distance 706, or each of electrodes 113 and 114 may be offset from face 502 by a different distance within the range of values for distance 706 as described above.

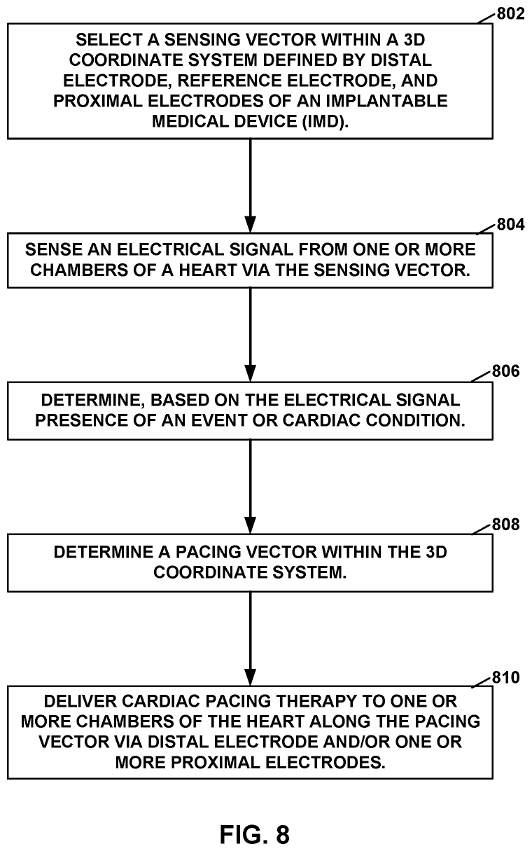

FIG. 8 is a flow diagram illustrating an example process for delivering cardiac pacing therapy to heart 102 of a patient via an example device of any of FIGS. 1-7. The technique of FIG. 8 will be described with concurrent reference to device 104 (FIG. 1) although a person having ordinary skill in the art will understand that the technique may be performed in reference to another implantable medical lead or other medical device. In addition, while the example process of FIG. 8 is described primarily with reference to an atrium of heart 102 as a first chamber and a ventricle of heart 102 as a second chamber, the example process described herein may be applied to other combinations of the chambers of heart 102. The example process described herein may be similarly performed on a 2D coordinate system with device 104 having one proximal electrode 114 (e.g., as illustrated in FIG. 2B).

Device 104 may select a sensing vector within a 3D coordinate system defined by distal electrode 112, reference electrode 113, and proximal electrodes 114 of implantable medical device (IMD) 104 (also referred to herein as “device 104”) (802). Each of distal electrode 112 and proximal electrodes 114 may define a reference axis 506 (e.g., Z-axis 506A, X-axis 506B, Y-axis 506C) with reference electrode 113 and may detect signal components of an electrical signal along the corresponding reference axis 506. For example, distal electrode 112 and reference electrode 113 defines Z-axis 506A and detects signal components of electrical signals in heart 102 (e.g., in target implant region 106) that are along Z-axis 506A. Together, reference axes 506 define a 3D coordinate system encompassing an area containing target implant region 106, distal electrode 112, reference electrode 113, and proximal electrode 114. Each of reference axes 506 may be orthogonal to every other reference axes 506, e.g., to prevent any of electrodes 112 and 114 from sensing overlapping signals along other reference axes 506. For example, when Z-axis 506A is orthogonal to X-axis 506B and Y-axis 506C, distal electrode 112 only senses signal components of electrical signals along Z-axis 506A and does not sense signal components of the electrical signals along X-axis 506B or Y-axis 506C. By using the 3D coordinate system, device 104 may sense electrical signals within target implant region 106 and determine characteristics of the electrical signals (e.g., EGMs, type of signal (i.e., atrial signal or ventricular signal)) without requiring device 104 to be implanted at a specific orientation within target implant region 106.

Device 104 may sense an electrical signal from one or more chambers of heart 102 along the sensing vector (804). Each sensing vector may be a combination of sensed signal components from two or more of distal electrode 112 and proximal electrodes 114. For example, a sensing vector may be a combination of sensed signal components from distal electrode 112 and proximal electrode 114A, from proximal electrode 114A and proximal electrode 114B, or from distal electrode 112 and proximal electrode 114B. Sensing circuitry 304 may detect and represent any sensed signal within the 3D coordinate system in terms of two or more sensing vectors. Sensing circuitry 304 may sense electrical signals along some sensing vectors to only sense electrical signals from a particular chamber of heart 102. For example, when device 104 is implanted within an atrium of heart 102 (e.g., the RA), sensing circuitry 304 may sense electrical signals from a ventricle of heart 102 (e.g., the LV) via a sensing vector along Z-axis 506A and X-axis 506B. Similarly, sensing circuitry 304 may sense electrical signals from the atrium of heart 102 via a sensing vector along X-axis 506B and Y-axis 506C. Sensing circuitry 304 may sense the electrical signal along one or more sensing vectors to determine a location (e.g., relative to one or more of distal electrode 112, reference electrode 113, and proximal electrodes 114), amplitude, and/or frequency of the electrical signal within heart 102. Sensing circuitry 304 may combine the sensed electrical signals along the one or more sensing vectors into a combined electrical signal corresponding to the actual electrical signal within heart 102.

Based on the sensed electrical signals (e.g., EGMs), device 104 (e.g., sensing circuitry 304 and/or processing circuitry 310) may determine the presence of an event or a cardiac condition (806). Cardiac conditions may include, but are not limited to, arrhythmias, tachycardia, or the like. Events may include, but are not limited to, atrial depolarization, ventricular depolarization, or the like. Device 104 may determine the presence of an event or cardiac condition and determine whether to deliver cardiac pacing therapy to one or more chambers of heart 102 based on the determination. In some examples, sensing circuitry 304 of device 104 makes the determination and transmit the results to processing circuitry 310 for determination of whether to deliver cardiac pacing therapy and/or parameters of the cardiac pacing therapy. In some examples, sensing circuitry 304 transmits the sensed electrical signals to processing circuitry 310 and processing circuitry 310 makes the determination based on the sensed electrical signals.

Based on a determination that device 104 should deliver cardiac pacing therapy to one or more chambers of heart 102, processing circuitry 310 may determine a pacing vector within the 3D coordinate system (808). A pacing vector may connect a target location for cardiac pacing with one or more of electrodes 112, 113, and 114. After device 104 selects the pacing vector, processing circuitry 310 may cause signal generation circuitry 306 to deliver cardiac pacing therapy to one or more chambers of heart 102 along the pacing vector via distal electrode 112 and/or one or more of proximal electrodes 114 (810). For example, device 104 may deliver cardiac pacing therapy to ventricular myocardium 108 via distal electrode 112 and reference electrode 113. In another example, device 104 may deliver cardiac pacing therapy to atrial myocardium 406 via one or more of proximal electrodes 114 and reference electrode 113. In some examples, device 104 selects an optimal proximal electrode of proximal electrodes 114 based at least in part of the pacing vector and/or the sensed electrical signals (e.g., on the sensed electrical signal components, on the sensed electrical signal along a sensing vector, or on combined electrical signals). Device 104 may then deliver cardiac pacing to one or more chambers of heart 102 via the optimal proximal electrode. In some examples, device 104 may deliver cardiac pacing therapy to one or more chambers of heart 102 (e.g., to the LA) via both proximal electrodes 114 simultaneously. In some examples, device 104 may alternate delivery of cardiac pacing therapy from proximal electrodes 114.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

In addition, it should be noted that system described herein may not be limited to treatment of a human patient. In alternative examples, the system may be implemented in non-human patients, e.g., primates, canines, equines, pigs, and felines. These other animals may undergo clinical or research therapies that may benefit from the subject matter of this disclosure.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Various examples have been described. These and other examples are within the scope of the following claims.

Example 1. A device comprising: an elongated housing that extends from a proximal end of the housing to a distal end of the housing, the elongated housing being configured to be implanted wholly within a first chamber of a heart, the first chamber of the heart having wall tissue; a distal electrode extending distally from the distal end of the elongated housing, the distal electrode being configured to penetrate into wall tissue of a second chamber of the heart that is separate from the first chamber of the heart; a reference electrode extending from the distal end of the elongated housing; one or more proximal electrodes extending from the distal end of the elongated housing, wherein the one or more proximal electrodes are separate from the distal electrode and the reference electrode; sensing circuitry within the elongated housing and coupled to the distal electrode, the reference electrode, and the one or more proximal electrodes; and processing circuitry within the elongated housing, the processing circuitry being configured to control the sensing circuitry to sense electrical signals of the heart via the distal electrode, the reference electrode, and the one or more proximal electrodes.

Example 2. The device of Example 1, wherein the first chamber comprises an atrium of the heart, and wherein the second chamber comprises a ventricle of the heart.

Example 3. The device of any of Examples 1 and 2, wherein the reference electrode and each proximal electrode of the one or more proximal electrodes are configured to maintain contact with the wall tissue of the first chamber without penetration of the wall tissue of the first chamber.

Example 4. The device of any of Examples 1-3, wherein the one or more proximal electrodes comprise a first proximal electrode, wherein the distal electrode and the reference electrode defines a distal axis, wherein the first proximal electrode and the reference electrode defines a proximal axis, and wherein the processing circuitry is configured to sense the electrical signals of the heart along a sensing vector within a plane defined by the distal axis and the proximal axis.

Example 5. The device of Example 4, wherein the distal axis is orthogonal to the proximal axis, and wherein the distal axis extends distally away from the distal end of the housing.

Example 6. The device of any of Examples 1-5, wherein the distal electrode and the reference electrode define a first axis, wherein a first proximal electrode of the one or more proximal electrodes and the reference electrode define a second axis, and wherein a second proximal electrode of the one or more proximal electrodes and the reference electrode define a third axis.

Example 7. The device of Example 6, wherein the processing circuitry is configured to: cause sensing circuitry within the elongated housing to sense, along a sensing vector, at least two of a first electrical signal via the first axis, a second electrical signal via the second axis, and a third electrical signal via the third axis; determine a fourth electrical signal based on the at least two of the first electrical signal, the second electrical signal, and the third electrical signal according to a sensing vector; and control the delivery of cardiac pacing therapy to one or more of the first chamber or the second chamber based on the fourth electrical signal.

Example 8. The device of Example 7, wherein the processing circuitry is configured to determine the fourth electrical signal based on the first electrical signal, the second electrical signal, the third electrical signal, and the sensing vector, wherein the sensing vector is a three-dimensional (3D) vector relative to the first axis, the second axis, and the third axis.

Example 9. The device of any of Examples 6-8, wherein the first axis, the second axis, and the third axis are orthogonal to each other.

Example 10. The he device of any of Examples 1-9, wherein each of the distal electrode and the one or more proximal electrodes is electrically connected to signal generation circuitry within the housing through a corresponding feedthrough assembly.

Example 11. The device of any of Examples 1-10, wherein the processing circuitry is configured to, prior to penetration of the wall tissue of the first chamber by the distal electrode: cause the sensing circuitry to sense electrical signals via the distal electrode, the reference electrode, and the one or more proximal electrodes; and determine, based on the sensed electrical signals, a position and orientation of the device within the first chamber of the heart.

Example 12. The device of any of Examples 1-11, wherein the distal electrode comprises an elongated body extending distally from the distal end of the elongated housing, the elongated body comprising: a helix having one or more coils; and a distal end configured to puncture the wall tissue of the first chamber and extend into the wall tissue of the second chamber.

Example 13. The device of Example 12, wherein the helix extends from a proximal end to a distal end, and wherein the distal end of the helix defines a larger outer diameter than the proximal end of the helix, and wherein the distal end of the elongated body and the reference electrode defines a distal axis substantially parallel to the longitudinal axis.

Example 14. The device of any of Examples 12 and 13, wherein the helix defines a varying diameter along a longitudinal axis of the helix.

Example 15. The device of any of Examples 1-14, wherein the distal end of the elongated housing further comprises one or more therapeutic substance dispensing devices.

Example 16. The device of Example 15, wherein at least one therapeutic substance dispensing device is disposed within a corresponding recess within the one or more proximal electrodes or within the reference electrode.

Example 17. The device of any of Examples 15 and 16, wherein the one or more therapeutic substance dispensing devices comprises one or more monolithic controlled release devices.

Example 18. The device of any of Examples 15-17, wherein the one or more therapeutic substance dispensing devices are disposed around a circumference of the distal end of the elongated housing.

Example 19. The device of any of Examples 1-18, further comprising one or more anti-rotation features disposed on the distal end of the housing and configured to prevent rotation of the device due to movement of the wall tissue of the first chamber.

Example 20. A method comprising: sensing, by sensing circuitry of an implantable medical device (IMD) and via a plurality of combinations of a distal electrode extending distally from a distal end of an elongated housing of the IMD, a reference electrode extending from the distal end of the elongated housing, and one or more proximal electrodes extending from the distal end of the elongated housing, a plurality of electrical signals from a heart; determining, by processing circuitry of the IMD and based on the sensed electrical signals and a sensing vector, a combined electrical signal; and delivering, by one or more of the distal electrode and the one or more proximal electrodes, cardiac pacing therapy to one or more chambers of the heart based on the combined signal, wherein the distal electrode is configured to penetrate into the wall tissue of the second chamber, and wherein the one or more proximal electrodes and the reference electrode are configured to maintain contact with the wall tissue of the first chamber without penetration of the wall tissue of the first chamber by the one or more proximal electrodes.

Example 21. The method of Example 20, where the distal electrode and the reference electrode define a first axis, wherein a first proximal electrode of the one or more proximal electrodes and the reference electrode define a second axis, and wherein a second proximal electrode of the one or more proximal electrodes and the reference electrode define a third axis.

Example 22. The method of Example 21, wherein sensing the electrical signal from the heart comprises: sensing, by the sensing circuitry, a first electrical signal via the first axis, a second electrical signal via the second axis, and a third electrical signal via the third axis.

Example 23. The method of Example 22, wherein determining the combined signal comprises: determining, by the processing circuitry, the combined signal based on the sensing vector and a combination of two or more of the first electrical signal, the second electrical signal, the third electrical signal, wherein the sensing vector is a three-dimensional (3D) vector relative to the first axis, the second axis, and the third axis.

Example 24. The method of any of Examples 21-23, wherein the first axis, the second axis, and the third axis are orthogonal relative to each other.

Example 25. The method of any of Examples 21-24, wherein the first axis is orthogonal to the distal end of the elongated housing.

Example 26. The method of any of Examples 21-25 wherein delivering the cardiac pacing therapy to the one or more chambers of the heart comprises: determining, by the processing circuitry and based on the combined signal, whether to deliver the cardiac pacing therapy to the first chamber of the heart; and delivering, based on a determination to deliver the cardiac pacing therapy to the first chamber and by one or more of the first proximal electrode and the second proximal electrode, the cardiac pacing therapy to the wall tissue of the first chamber.

Example 27. The method of Example 26, wherein delivering the cardiac pacing therapy to the first chamber further comprises alternatively delivering, by the first proximal electrode and the second proximal electrode, the cardiac pacing therapy to the wall tissue of the first chamber.

Example 28. The method of any of Examples 20-27, wherein the distal electrode comprises an elongated body extending distally from the distal end of the elongated housing, the elongated body comprising: a helix having one or more coils; and a distal end configured to puncture the wall tissue of the first chamber and extend into the wall tissue of the second chamber.

Example 29. A device comprising: an elongated housing extending from a proximal end to a distal end, the elongated housing being configured to be implanted wholly within a first chamber of a heart; a distal electrode extending distally from the distal end of the elongated housing, the distal electrode being configured to penetrate into wall tissue of a second chamber of the heart, the second chamber of the heart being separated from the first chamber of the heart; two proximal electrodes extending from the distal end of the elongated housing, wherein the two proximal electrodes are separate from the distal electrode, and wherein each proximal electrode of the two proximal electrodes is configured to maintain contact with wall tissue of the first chamber without penetration of the wall tissue of the first chamber; a reference electrode extending from the distal end of the elongated housing and separate from the distal electrode and the two or more proximal electrodes, the reference electrode being configured to: define a first axis with the distal electrode; define a second axis with a first proximal electrode of the two proximal electrodes; and define a third axis with a second proximal electrode of the two proximal electrodes; sensing circuitry disposed within the elongated housing; signal generation circuitry disposed within the elongated housing; and processing circuitry within the elongated housing, the processing circuitry being configured to: cause the sensing circuitry to sense electrical signals from the heart along at least two of the first axis, the second axis, and the third axis via the distal electrode, the first proximal electrode, and the second proximal electrode, respectively; determine, based on the sensed electrical signals and a three-dimensional (3D) sensing vector, a combined signal; and cause the signal generation circuitry to deliver cardiac pacing therapy to the heart via one or more of the distal electrode, the first proximal electrode, or the second proximal electrode based on the combined electrical signal.

Example 30. The device of Example 29, wherein the processing circuitry is further configured to: determine a morphology of a wave of the combined signal; and cause the signal generation circuitry to deliver the cardiac pacing therapy to the heart based on the determined morphology of the wave.

Example 31. The device of any of Examples 29 and 30, wherein the processing circuitry is further configured to determine a presence of a cardiac condition based on the combined signal.

Example 32. The device of Example 31, wherein the cardiac condition comprises tachycardia.

Example 33. The device of any of Examples 29-32, wherein the first chamber comprises an atrium of the heart, and wherein the second chamber comprises a ventricle of the heart.

Example 34. The device of any of Examples 29-33, wherein the 3D sensing vector is defined relative to the first axis, the second axis, and the third axis.

Example 35. The device of any of Examples 29-34, wherein each of the distal electrode and the two proximal electrodes is electrically connected to the signal generation circuitry through a corresponding feedthrough assembly.

Example 36. The device of any of Examples 29-35 wherein the first axis, the second axis, and the third axis are orthogonal to each other.

Example 37. The device of any of Examples 29-36, wherein the distal electrode comprises an elongated body extending distally from the distal end of the elongated housing, the elongated body comprising: a helix having one or more coils; and a distal end configured to puncture the wall tissue of the first chamber and extend into the wall tissue of the second chamber.

Example 38. The device of Example 37, wherein the helix extends from a proximal end to a distal end, wherein the helix defines a larger outer diameter at the distal end than at the proximal end, and wherein the distal end of the elongated body and the reference electrode define a distal axis substantially parallel to the longitudinal axis.

Example 39. The device of Example 38, wherein the distal end of the helix is disposed at a same circumferential and radial position as the reference electrode relative to the longitudinal axis.

Example 40. The device of any of Examples 37-39, wherein the helix defines a varying diameter along a longitudinal axis of the helix.

1. A device comprising:
an elongated housing that extends from a proximal end of the elongated housing to a distal end of the elongated housing, the elongated housing being configured to be implanted wholly within a first chamber of a heart, the first chamber of the heart having wall tissue;
a distal electrode extending distally from the distal end of the elongated housing, the distal electrode being configured to penetrate into wall tissue of a second chamber of the heart that is separate from the first chamber of the heart;
a reference electrode extending from the distal end of the elongated housing;
one or more proximal electrodes extending from the distal end of the elongated housing, wherein the one or more proximal electrodes are separate from the distal electrode and the reference electrode;
sensing circuitry disposed within the elongated housing and coupled to the distal electrode, the reference electrode, and the one or more proximal electrodes; and
processing circuitry disposed within the elongated housing, the processing circuitry being configured to control the sensing circuitry to sense electrical signals of the heart via the distal electrode, the reference electrode, and the one or more proximal electrodes.

2. The device of claim 1, wherein the first chamber comprises an atrium of the heart, and wherein the second chamber comprises a ventricle of the heart.

3. The device of claim 1, wherein the reference electrode and each proximal electrode of the one or more proximal electrodes are configured to maintain contact with the wall tissue of the first chamber without penetration of the wall tissue of the first chamber.

4. The device of claim 1, wherein the one or more proximal electrodes comprise a first proximal electrode, wherein the distal electrode and the reference electrode defines a distal axis extending distally away from the distal end of the elongated housing, wherein the first proximal electrode and the reference electrode defines a proximal axis orthogonal to the distal axis, and wherein the processing circuitry is configured to sense the electrical signals of the heart along a sensing vector within a plane defined by the distal axis and the proximal axis.

5. (canceled)

6. The device of claim 1, wherein the distal electrode and the reference electrode define a first axis, wherein a first proximal electrode of the one or more proximal electrodes and the reference electrode define a second axis, and wherein a second proximal electrode of the one or more proximal electrodes and the reference electrode define a third axis.

7. The device of claim 6, wherein the processing circuitry is configured to:
cause sensing circuitry within the elongated housing to sense, along a sensing vector, at least two of a first electrical signal along the first axis, a second electrical signal along the second axis, and a third electrical signal along the third axis;
determine a fourth electrical signal based on the at least two of the first electrical signal, the second electrical signal, and the third electrical signal according to the sensing vector; and
control the delivery of cardiac pacing therapy to one or more of the first chamber or the second chamber based on the fourth electrical signal.

8. The device of claim 7, wherein the processing circuitry is configured to determine the fourth electrical signal based on the first electrical signal, the second electrical signal, the third electrical signal, and the sensing vector, wherein the sensing vector is a three-dimensional (3D) vector relative to the first axis, the second axis, and the third axis.

9. The device of claim 6, wherein the first axis, the second axis, and the third axis are orthogonal to each other.

10. (canceled)

11. The device of claim 1, wherein the processing circuitry is configured to, prior to penetration of the wall tissue of the first chamber by the distal electrode:
cause the sensing circuitry to sense electrical signals via the distal electrode, the reference electrode, and the one or more proximal electrodes; and
determine, based on the sensed electrical signals, a position and orientation of the device within the first chamber of the heart.

12. The device of claim 1, wherein the distal electrode comprises an elongated body extending distally from the distal end of the elongated housing, the elongated body comprising:
a helix having one or more coils; and
a distal end configured to puncture the wall tissue of the first chamber and extend into the wall tissue of the second chamber.

13. The device of claim 12, wherein the helix extends from a proximal end to a distal end, and wherein the distal end of the helix defines a larger outer diameter than the proximal end of the helix, and wherein the distal end of the elongated body and the reference electrode defines a distal axis substantially parallel to the longitudinal axis.

14-15. (canceled)

16. A method comprising:
sensing, by sensing circuitry of an implantable medical device (IMD) and via a plurality of combinations of a distal electrode extending distally from a distal end of an elongated housing of the IMD, a reference electrode extending from the distal end of the elongated housing, and one or more proximal electrodes extending from the distal end of the elongated housing, a plurality of electrical signals from a heart;
determining, by processing circuitry of the IMD and based on the sensed electrical signals and a sensing vector, a combined electrical signal; and
delivering, by one or more of the distal electrode and the one or more proximal electrodes, cardiac pacing therapy to one or more chambers of the heart based on the combined signal,
wherein the distal electrode is configured to penetrate into the wall tissue of the second chamber, and wherein the one or more proximal electrodes and the reference electrode are configured to maintain contact with the wall tissue of the first chamber without penetration of the wall tissue of the first chamber by the one or more proximal electrodes.

17. The method of claim 16,
where the distal electrode and the reference electrode define a first axis, wherein a first proximal electrode of the one or more proximal electrodes and the reference electrode define a second axis, wherein a second proximal electrode of the one or more proximal electrodes and the reference electrode define a third axis, and
wherein sensing the electrical signal from the heart comprises:
sensing, by the sensing circuitry, a first electrical signal via the first axis, a second electrical signal via the second axis, and a third electrical signal via the third axis.

18. The method of claim 17, wherein determining the combined signal comprises:
determining, by the processing circuitry, the combined signal based on the sensing vector and a combination of two or more of the first electrical signal, the second electrical signal, the third electrical signal,
wherein the sensing vector is a three-dimensional (3D) vector relative to the first axis, the second axis, and the third axis.

19. The method of claim 17, wherein the first axis, the second axis, and the third axis are orthogonal relative to each other.

20. The method of claim 16, wherein delivering the cardiac pacing therapy to the one or more chambers of the heart comprises:
determining, by the processing circuitry and based on the combined signal, whether to deliver the cardiac pacing therapy to the first chamber of the heart; and
delivering, based on a determination to deliver the cardiac pacing therapy to the first chamber and by one or more of the first proximal electrode and the second proximal electrode, the cardiac pacing therapy to the wall tissue of the first chamber.

21. The method of claim 20, wherein delivering the cardiac pacing therapy to the first chamber further comprises alternatively delivering, by the first proximal electrode and the second proximal electrode, the cardiac pacing therapy to the wall tissue of the first chamber.

22. A device comprising:
an elongated housing extending from a proximal end to a distal end, the elongated housing being configured to be implanted wholly within a first chamber of a heart;
a distal electrode extending distally from the distal end of the elongated housing, the distal electrode being configured to penetrate into wall tissue of a second chamber of the heart, the second chamber of the heart being separated from the first chamber of the heart;
two proximal electrodes extending from the distal end of the elongated housing, wherein the two proximal electrodes are separate from the distal electrode, and wherein each proximal electrode of the two proximal electrodes is configured to maintain contact with wall tissue of the first chamber without penetration of the wall tissue of the first chamber;
a reference electrode extending from the distal end of the elongated housing and separate from the distal electrode and the two or more proximal electrodes, the reference electrode being configured to:
define a first axis with the distal electrode;
define a second axis with a first proximal electrode of the two proximal electrodes; and
define a third axis with a second proximal electrode of the two proximal electrodes;
sensing circuitry disposed within the elongated housing;
signal generation circuitry disposed within the elongated housing; and
processing circuitry within the elongated housing, the processing circuitry being configured to:
cause the sensing circuitry to sense electrical signals from the heart along at least two of the first axis, the second axis, and the third axis via the distal electrode, the first proximal electrode, and the second proximal electrode, respectively;
determine, based on the sensed electrical signals and a three-dimensional (3D) sensing vector, a combined signal; and
cause the signal generation circuitry to deliver cardiac pacing therapy to the heart via one or more of the distal electrode, the first proximal electrode, or the second proximal electrode based on the combined electrical signal.

23. The device of claim 22, wherein the processing circuitry is further configured to:
determine a morphology of a wave of the combined signal; and
cause the signal generation circuitry to deliver the cardiac pacing therapy to the heart based on the determined morphology of the wave.

24. The device of claim 22, wherein the 3D sensing vector is defined relative to the first axis, the second axis, and the third axis.
